(19) United States
(12) Patent Application Publication
(10) Pub. No.: US 2024/0197683 A1
(43) Pub. Date: Jun. 20, 2024

(54) USE OF ERGOTHIONEINE (EGT) FOR PREVENTION AND TREATMENT OF SEPSIS

(71) Applicant: JIANGSU UNIVERSITY, Zhenjiang (CN)

(72) Inventor: Zhimin TAO, Zhenjiang (CN)

(21) Appl. No.: 18/465,252

(22) Filed: Sep. 12, 2023

(30) Foreign Application Priority Data
May 16, 2023 (CN) 202310557089.0

(51) Int. Cl. A61K 31/4164; A61P 31/00; B82Y 5/00

(52) U.S. Cl. CPC A61K 31/4164; A61P 31/00; B82Y 5/00

(57) ABSTRACT

The present disclosure discloses use of ergothioneine (EGT) for the prevention or treatment of sepsis. The present disclosure studies the anti-inflammatory mechanism of ergothioneine in sepsis, aiming to provide a therapeutically effective dose and reveal the signaling pathway ergothioneine probably acts on in the treatment of sepsis, and provide experimental data support for the understanding of the mechanism behind the systemic inflammatory reactions of sepsis and for the use of ergothioneine as a new drug for treatment. The present disclosure also provides ergothioneine (EGT) loaded on a nanocarrier, and use thereof in the prevention or treatment of sepsis.

The present application claims priority to Chinese Patent Application No. 2023105570890 filed with China National Intellectual Property Administration on May 16, 2023, entitled “USE OF ERGOTHIONEINE (EGT) FOR PREVENTION AND TREATMENT OF SEPSIS”, which is incorporated herein by reference in its entirety.

### TECHNICAL FIELD

The present disclosure relates to the technical field of pharmaceuticals, and particularly to use of ergothioneine (EGT) in the prevention and treatment of sepsis.

### BACKGROUND

Sepsis has recently been redefined as life-threatening organ dysfunction caused by the host's dysregulated response to infection. For decades, it has been a highly fatal disease, with reliable diagnostic and therapeutic decisions far from optimal. At present, the morbidity of sepsis is estimated to be 270 cases per 100 thousand people per year, and the mortality is about 26%; 30%-50% of the patients in intensive care units (ICUs) die of it after admission, and it has caused high diagnostic and therapeutic costs. Septic shock patients are a subpopulation of sepsis patients with circulatory, cellular and/or metabolic dysfunction and an increased risk of death (>25%). Activities such as Surviving Sepsis Campaign and World Sepsis Day have increased people's awareness of the global sepsis burden and related diagnoses and treatments. Most of the clinical trials of sepsis have now failed. A better understanding of the pathophysiological process of sepsis is a key to upsetting the status quo and significantly improving sepsis prognosis.

The complex signaling pathways and the host'dysregulated response make sepsis a life-threatening heterogeneous syndrome distinct from mild infection. Sepsis is very pathophysiologically complex. The typical pathophysiological process of sepsis can be summarized as a dynamic process that develops continuously, i.e., systemic inflammatory response syndrome (SIRS), sepsis, severe sepsis, septic shock and multiple organ dysfunction syndrome. Sepsis originates from the host's recognition of microbial-derived pathogen-associated molecular patterns (PAMPs) or endogenous damage-associated molecular patterns (DAMPs). They are recognized by a range of pattern recognition receptors (PRRs) located on the cell membrane or in the intracellular space, and the recognition leads to activation of intracellular signaling pathways. There is evidence that several PAMPs can stimulate TLR4. These molecules include the lipopolysaccharide (LPS) from gram-negative bacteria, the fusion (F) protein from the respiratory syncytial virus (RSV), and the envelope protein from the mouse mammary tumor virus (MMTV). LPS is the major component of the outer membrane of gram-negative bacteria. Its chemical structure includes three parts: the most conserved lipid A, the core oligosaccharide and the O antigen. As an endotoxin, it exhibits various biological activities when acting on humans or animals. It functions mainly via TLR4 present on the cell membrane surface of host cells. The outer LPS is recognized by the TLR4-MD2 complex and CD14 protein located on the cell membrane. CD14 protein specifically recognizes lipid A of LPS and then transduces signals via MyD88 and TRIF to activate NF-κB- and IRF3-mediated transcription of the immune system genes (including cytokines and chemokines). The overactivation of TLR4 by LPS and the consequent cytokine storm are believed to be the basis of sepsis.

Ergothioneine (EGT) is a unique sulfur-containing crystal compound separated in 1909. It is a natural-occurring rare amino acid. It is chemically stable and has significant antioxidant capacity. It has very wide biological and pharmaceutical application prospects. As a strong antioxidant, it has a number of physiological functions such as effectively eliminating free radicals in vivo, maintaining DNA synthesis and normal cell growth, etc., has the effect of whitening skin, resisting aging and radiation, etc., and also has prophylactic and inhibitory effects on the majority of diseases caused by free radical damage, such as immune inflammation, cancer, etc. EGT accumulates preferentially in organs, cells and secretions in the body. These organs, cells and secretions, such as the liver, the kidneys, red blood cells, the lens and semen, are prone to high levels of oxidative stress and inflammation. EGT has many advantages in antioxidant activity over other antioxidants such as glutathione and ascorbic acid. First, as a natural antioxidant, EGT can accumulate in millimolar concentrations in certain tissues without toxicity. According to an analysis of the source and usage level of EGT, the total EGT intake is 1.7 mg/kg body weight per day for adults and 3.7 mg/kg body weight per day for children. High-dose or acute experiments in animals and cells showed no toxic effects of EGT. For example, 5,000 mg/mL EGT did not cause genotoxicity and chromosomal aberration in a study with hamster lung cells. Large-amount and acute (2 weeks) or long-term (90 days) oral EGT supplement (0.9% concentration) experiments on rats showed no reproductive and developmental toxicity. Data show that EGT can significantly reduce the expression of proinflammatory cytokines IL-1β, IL-6, IL-8, TNF-α and cycloxygenase 2 (COX2) mRNA and COX2 activity. Co-treatment of A549 cells with ergothioneine and either TNF-α or H₂O₂ inhibited NF-κB activation and protected the cells from decreases in the GSH level, suggesting that ergothioneine inhibited NF-κB activation through a mechanism dependent on its thiol-mediated antioxidant properties. In addition, EGT can reduce acute lung injury and, by inhibiting oxidation, reduce inflammatory stress, TNF-α-induced NF-κB activation and IL-8 release into alveolar epithelial cells in cytokine-infused rats. EGT can also significantly ameliorate colon length shortening and pathological injury to the colon, and can mediate proinflammatory factors and inhibit the TLR-4/MyD88/NF-κB signaling pathway by down-regulating expression.

In sepsis, the TLR-4/MyD88/NF-κB signaling pathway and the oxidative stress pathway are activated and inflammatory factors are released, causing multiple organ dysfunction. Ergothioneine can inhibit NF-κB activation and is therefore presumed to regulate the activation of the TLR-4/MyD88/NF-κB signaling pathway and inhibit oxidative stress, reducing inflammatory factors such as IL-6 and TNF-α proinflammatory factors, etc., and thereby alleviating multiple organ dysfunction in mice with sepsis.

### SUMMARY

A first aspect of the present disclosure provides use of ergothioneine and/or a pharmaceutically acceptable derivative thereof for manufacturing a drug for the treatment or prevention of sepsis or related disease. More specifically, the present disclosure relates to use of ergothioneine and/or a pharmaceutically acceptable derivative thereof for manufacturing a drug for the treatment or prevention of sepsis in a subject, wherein the drug comprises a therapeutically or prophylactically effective amount of ergothioneine and/or the pharmaceutically acceptable derivative thereof. More specifically, the subject is a human.

A second aspect of the present disclosure provides a method of treatment or prevention of sepsis or related disease, the method comprising administering ergothioneine and/or a pharmaceutically acceptable derivative thereof. More specifically, the present disclosure relates to a method of treatment or prevention of sepsis in a subject, the method comprising administering to the subject a therapeutically or prophylactically effective amount of ergothioneine and/or the pharmaceutically acceptable derivative thereof. More specifically, the subject is a human.

A third aspect of the present disclosure provides use of ergothioneine and/or a pharmaceutically acceptable derivative thereof for the treatment or prevention of sepsis or related disease. More specifically, the present disclosure relates to use of ergothioneine and/or the pharmaceutically acceptable derivative thereof for the treatment or prevention of sepsis in a subject, which comprises administering to the subject a therapeutically or prophylactically effective amount of ergothioneine and/or the pharmaceutically acceptable derivative thereof. More specifically, the subject is a human. A fourth aspect of the present disclosure provides ergothioneine and/or a pharmaceutically acceptable derivative thereof for use in the treatment or prevention of sepsis or related disease. More specifically, the present disclosure relates to ergothioneine and/or the pharmaceutically acceptable derivative thereof for use in the treatment or prevention of sepsis in a subject, which comprises administering to the subject a therapeutically or prophylactically effective amount of ergothioneine and/or the pharmaceutically acceptable derivative thereof. More specifically, the subject is a human.

In a specific embodiment of the present disclosure, the sepsis-related disease includes septic shock or sepsis-induced organ injury such as lung injury, intestinal injury, liver injury and kidney injury.

In a specific embodiment of the present disclosure, the treatment or prevention includes amelioration of a disease symptom or improvements in health in a subject, including an increase in survival rate, extension of the subject's survival, a reduction in inflammatory factor release, etc.

In a specific embodiment of the present disclosure, the treatment or prevention alleviates lung inflammation, alleviates pulmonary edema, protein leakage and vascular damage, and improves lung oxygenation.

In a specific embodiment of the present disclosure, the ergothioneine and/or the pharmaceutically acceptable derivative thereof treat(s) or prevent(s) sepsis or the related disease by inhibiting phosphorylation activation of the TLR-4/MyD88/NF-κB signaling pathway.

In a specific embodiment of the present disclosure, the ergothioneine and/or the pharmaceutically acceptable derivative thereof are/is used concurrently with one or more additional drugs for the treatment or prevention of sepsis or the related disease.

Preferably, the drug is administered by intraperitoneal injection, intramuscular injection or intravenous injection, or is administered orally.

Preferably, the drug is administered in an amount of 0.1-150 mg/kg, preferably 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145 or 150 mg/kg, on an ergothioneine basis.

Preferably, the drug is a solid or liquid preparation in unit dosage form, such as a capsule, a pill, a tablet, a lozenge, a troche, a collosol, a powder, a solution, a suspension or an emulsion.

Preferably, the content of ergothioneine or the pharmaceutically acceptable salt thereof in the unit dosage form is 0.1-1000 mg, preferably 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950 or 1000 mg, on an ergothioneine basis.

Preferably, the nano-sized pharmaceutical carrier loaded with ergothioneine is used to treat or prevent sepsis. The nano-sized pharmaceutical carrier includes organic nanoparticles, inorganic nanoparticles and nanoparticles of natural origin. The organic nanoparticles include, but are not limited to, liposomes, micelles, emulsions and solid lipid nanoparticles (SLNs) and polymeric nanoparticles (PNPs); the inorganic nanoparticles (INPs) are synthesized from inorganic particles and biodegradable polycations, and include, but are not limited to, metals, metal oxides, carbon materials, and magnetic nanoparticles consisting of superparamagnetic iron oxide nanoparticles (SPIONs); the nanoparticles of natural origin include extracellular vesicles (EVs) actively released by cells and nanovesicles (NVs) prepared by artificial extrusion, wherein EVs are diverse, nano-sized membrane vesicles actively released by cells; similarly sized vesicles can be further classified according to biogenesis, size and biophysical properties thereof (e.g., exosomes, microvesicles, etc.).

A fifth aspect of the present disclosure provides a nanodrug, the drug comprising a nano-sized pharmaceutical carrier, and ergothioneine and/or a pharmaceutically acceptable derivative thereof loaded on the carrier. The nano-sized pharmaceutical carrier includes organic nanoparticles, inorganic nanoparticles and nanoparticles of natural origin. The organic nanoparticles include, but are not limited to, liposomes, micelles, emulsions and solid lipid nanoparticles (SLNs) and polymeric nanoparticles (PNPs); the inorganic nanoparticles (INPs) are synthesized from inorganic particles and biodegradable polycations, and include, but are not limited to, metals, metal oxides, carbon materials, and magnetic nanoparticles consisting of superparamagnetic iron oxide nanoparticles (SPIONs); the nanoparticles of natural origin include extracellular vesicles (EVs) actively released by cells and nanovesicles (NVs) prepared by artificial extrusion, wherein EVs are diverse, nano-sized membrane vesicles actively released by cells; similarly sized vesicles can be further classified according to biogenesis, size and biophysical properties thereof (e.g., exosomes, microvesicles, etc.).

### Definitions

Ergothioneine or a salt thereof that can be used may be a chemically synthesized product or a purified extract of a natural product. Golden/yellow oyster mushrooms (Pleurotus cornucopiae var. citrinopileatus) of the genus Pleurotus of the family Pleurotaceae are rich in ergothioneine. Ergothioneine is also present in mushrooms such as common mushrooms (Agaricus bisporus) including white mushroom, cremini mushroom and portabella mushroom, grey oyster mushroom (Pleurotus ostreatus), shiitake (Lentinula edodes), hen-of-the-wood (Grifola Frondosa), reishi mushroom (Ganoderma lucidum), lion's mane mushroom (Hericium erinaceus), Yanagi-matsutake (Agrocybe aegerita), girolle (Cantharellus cibarius), porcini (Boletus edulis), and morel (Morchella esculenta). When ergothioneine is obtained from a natural product, it is preferably extracted from a golden/yellow oyster mushroom, etc. Ergothioneine or a salt thereof may also be produced by microbial fermentation. An extract containing ergothioneine or a salt thereof produced by microbial fermentation or a purified product thereof may also be used. Extraction and purification from natural products, etc., can be performed by well-known methods. Ergothioneine or a salt thereof may be an isolated substance. Ergothioneine or a salt thereof produced by microbial fermentation is preferably used in the present disclosure.

The term “sepsis” includes different stages of the pathophysiological process of sepsis and related diseases arising therefrom, including but not limited to systemic inflammatory response syndrome (SIRS), sepsis, severe sepsis, septic shock, multiple organ dysfunction syndrome or multiple organ injury, etc.

The term “treatment or prevention” refers to a method for obtaining a beneficial or desired clinical result. For purposes of the present disclosure, the beneficial or desired clinical result includes, but is not limited to, alleviation of symptoms, diminishment of the extent of disease, stabilization of (i.e., not worsening) the state of disease, delaying or slowing of disease progression, amelioration or palliation of the state of disease, and remission (partial or total), whether detectable or undetectable. “Treatment” may also mean extending survival as compared to expected survival if not receiving treatment or therapy. Thus, “treatment” is an intervention intended to alter the pathology of a condition. Specifically, the treatment may directly prevent, slow down or decrease the pathology of cellular degeneration or damage, e.g., the pathology of organ cells in sepsis, or may render the cells more sensitive to treatment or therapy using other therapeutic agents.

The term “therapeutically or prophylactically effective amount”, “effective amount” or “sufficient amount” refers to an amount of an active agent sufficient to cause a particular biological state, effect and/or response, and specifically, in the present disclosure, refers to an amount sufficient to achieve the desired results when administered to a subject, including a mammal, such as a human, for example, an amount effective in treating sepsis. The effective amount of an agent described herein may vary depending on factors such as the state of disease, age, sex and body weight of the subject. As will be understood by those skilled in the art, dosages or treatment regimens may be adjusted to provide optimal therapeutic response. For example, in the present disclosure, ergothioneine and/or the pharmaceutically acceptable derivative thereof are/is administered at a dose of 0.1-150 mg/kg, preferably 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145 or 150 mg/kg, based on ergothioneine.

In addition, a therapeutically effective amount of a subject's treatment regimen may consist of a single administration or include a series of applications. The length of the treatment period depends on various factors such as the severity of the disease, the age of the subject, the concentration of the agent, the patient's response to the agent, or a combination thereof. It will also be understood that the effective dosage of the agent for treatment may be increased or decreased during a specific treatment regimen. Dose changes can be produced and become apparent by standard diagnostic analysis known in the art. In one aspect, the agent of the present disclosure can be administered before, during or after treatment with conventional therapies for the disease or condition in question, such as sepsis.

The term “subject” refers to any member of the animal kingdom, typically a mammal. The term “mammal” refers to any animal classified as a mammal, including humans, other higher primates, livestock and farm animals, as well as zoo, sport or pet animals such as dogs, cats, cows, horses, sheep, pigs, goats, rabbits, etc. Typically, the mammal is a human.

The term “pharmaceutically acceptable derivative” includes one or more of a pharmaceutically acceptable salt of ergothioneine, a tautomer of ergothioneine and a stereoisomer of ergothioneine.

The term “pharmaceutically acceptable salt” refers to salts that are suitable for use in contact with human or animal tissues without undue toxicity, irritation, allergic response, etc. within the scope of sound medical judgment and provide reasonable benefit/risk ratio. “Pharmaceutically acceptable salt” means any at least substantially non-toxic salt or ester salt of the compound of the present disclosure which, upon administration to a recipient, can directly or indirectly provide the compound of the present disclosure or a metabolite or residue thereof with inhibitory activity.

Pharmaceutically acceptable salts are well known in the art. Pharmaceutically acceptable salts of the compound disclosed herein include those derived from suitable inorganic and organic acids. Examples of pharmaceutically acceptable non-toxic acid addition salts are amino salts which are formed with inorganic acids such as hydrochloric acid, hydrobromic acid, phosphoric acid, sulfuric acid and perchloric acid, or with organic acids such as acetic acid, oxalic acid, maleic acid, tartaric acid, citric acid, succinic acid or malonic acid, or formed by other methods in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentane propionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, laurylsulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, etc.

### Formulation

The ergothioneine of the present disclosure may be used in any effective conventional dosage unit form including immediate release, sustained release, and timed release formulations, and ergothioneine may be administered together with a pharmaceutically acceptable carrier well known in the art in the following manner: oral, parenteral, topical, nasal, eye, intraperitoneal, intramuscular, intravenous, etc.

For oral administration, the compound may be formulated into solid or liquid preparations, such as a capsule, a pill, a tablet, a lozenge, a troche, a collosol, a powder, a solution, a suspension or an emulsion, and may be prepared according to methods known in the art for preparing pharmaceutical compositions. The solid unit dosage form may be a capsule, which may be of the ordinary hard or soft capsule form and contain, for example, surfactants, lubricants, and inert fillers such as lactose, sucrose, calcium phosphate and corn starch.

In another embodiment, ergothioneine, together with a conventional tablet base (e.g., lactose, sucrose and corn starch), can be compressed into a tablet in combination with the following substances: a binder (e.g., acacia, corn starch or gelatin), a disintegrant (e.g. potato starch, alginic acid, corn starch and guar gum, tragacanth gum and acacia) used to assist in disintegration and dissolution of the tablet after administration, a lubricant (e.g., talc, stearic acid or magnesium stearate, calcium stearate or zinc stearate) used to increase the flowability of the tablet granulate and prevent the tablet material from adhering to the surface of the tablet mold and the punch, a dye, a colorant, and a flavoring agent (e.g., peppermint oil, wintergreen oil or cherry flavor) used to improve the sensory properties of the tablets and render them more acceptable to patients. Suitable excipients for oral liquid dosage forms include dicalcium phosphate and diluents (such as water and alcohols (e.g., ethanol, benzyl alcohol and polyethylene glycol)), with or without the addition of pharmaceutically acceptable surfactants, suspending agents or emulsifiers. Various other substances may be present as coatings or otherwise used to alter the physical form of the dosage unit. For example, tablets, pills or capsules may be coated with shellac, sugar or both.

Dispersible powders and granules are suitable for the preparation of aqueous suspensions. They provide the active ingredient in admixture with a dispersing or wetting agent, a suspending agent and one or more preservatives. Examples of suitable dispersing or wetting agents and suspending agents are those mentioned above. Additional excipients may also be present, such as those sweeteners, flavoring agents and colorants described above.

Ergothioneine may also be administered parenterally at an injectable dose of the compound, i.e., subcutaneously, intravenously, intraocularly, intrasynovially, intramuscularly or intraperitoneally. The injectable dose is preferably in a physiologically acceptable diluent containing a pharmaceutical carrier. The pharmaceutical carrier can be a sterile liquid or a mixture of liquids. The liquid is, for example, water, saline, aqueous glucose and related sugar solutions, alcohols (e.g., ethanol, isopropanol or cetyl alcohol), diols (such as propylene glycol or polyethylene glycol), glycerol ketals (such as 2,2-dimethyl-1,1-dioxolane-4-methanol), ethers (such as polyethylene glycol (400)), oils, fatty acids, fatty acid esters or fatty acid glycerides or acetylated fatty acid glycerides. The diluent contains or does not contain additional pharmaceutically acceptable surfactants (such as soaps or detergents), suspending agents (such as pectin, carbomer, methylcellulose, hydroxypropyl methylcellulose or carboxymethylcellulose), or emulsifiers and other pharmaceutical adjuvants.

Exemplary oils that can be used in the parenteral preparations of the present disclosure are those derived from petroleum, animal, vegetable or synthetic sources, such as peanut oil, soybean oil, sesame oil, cottonseed oil, corn oil, olive oil, petrolatum and mineral oil. Suitable fatty acids include oleic acid, stearic acid, isostearic acid and myristic acid. Suitable fatty acid esters are, for example, ethyl oleate and isopropyl myristate. Suitable soaps include alkali metal, ammonium and triethanolamine salts of fatty acids, and suitable detergents include cationic detergents such as dimethyl dialkyl ammonium halides, alkylpyridinium halides and alkylamine acetate; anionic detergents such as alkylsulfonates, arylsulfonates and olefin sulfonates, alkylsulfates and alkylsulfosuccinates, olefin sulfates and olefin sulfosuccinates, ether sulfates and ether sulfosuccinates, as well as monoglyceride sulfates and monoglyceride sulfosuccinates; nonionic detergents such as fatty amine oxides, fatty acid alkanolamides and poly (oxyethylene-oxypropylene) or ethylene oxide copolymers or alkylene oxide copolymers; and amphoteric detergents such as alkyl-β-aminopropionates and 2-alkylimidazolinium quaternary ammonium salts, and mixtures thereof.

Exemplary surfactants for parenteral preparations are polyethylene sorbitan fatty acid esters (e.g., sorbitan monooleate) and a high-molecular-weight copolymers of ethylene oxide with a hydrophobic matrix (formed by condensation of propylene oxide and propylene glycol).

The sterile injectable preparation may also be a sterile solution or suspension for injection in a non-toxic parenterally acceptable diluent or solvent. Diluents and solvents that can be used are, for example, water, Ringer's solution, isotonic sodium chloride solution and isotonic glucose solution. In addition, sterile, fixed oils are generally used as a solvent or suspending medium. In this regard, any bland, fixed oil may be used, including synthetic monoglycerides or diglycerides. In addition, fatty acids (such as oleic acid) can be used in the preparation of injections.

For the dosage forms described above, the content of ergothioneine or the pharmaceutically acceptable salt thereof in the unit dosage form is 0.1-1000 mg, preferably 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950 or 1000 mg, based on ergothioneine.

### Therapeutic Kit

In other embodiments, the present disclosure also relates to a kit for convenient and efficient implementation of the method or use according to the present disclosure. Typically, the drug pack or kit comprises one or more containers filled with ergothioneine or the pharmaceutically acceptable salt thereof of the present disclosure. Such a kit is particularly suitable for delivery in solid oral forms such as tablets or capsules. Such a kit preferably comprises many unit doses, and may also comprise cards having a dose scheduled in their intended application order.

### Beneficial Effects

Although the inflammatory reactions of sepsis cause functional damage to multiple organs has attracted wide attention, there have been no relevant studies on using ergothioneine to treat and prevent septic multiple organ injury in either China or other countries. Ergothioneine, as an antioxidant, is also becoming well-known for its anti-inflammatory effect. However, there have been no studies on its effect on sepsis in either China or other countries. For the first time, the present disclosure studies the anti-inflammatory mechanism of ergothioneine in sepsis, aiming to provide a therapeutically effective dose and reveal the signaling pathway ergothioneine possibly acts on in the treatment of sepsis. The present disclosure provides experimental data support for the understanding of the mechanism behind the systemic inflammatory reactions of sepsis and for the use of ergothioneine as a new drug for treatment, and provide a new idea for clinical treatment.

Through experiments and analysis, the present disclosure finds that ergothioneine has a certain effect in treating sepsis diseases. It can be widely used as a drug for the clinical treatment of sepsis.

### BRIEF DESCRIPTION OF THE DRAWINGS

FIG. 1: Survival curves of mice with sepsis induced by different concentrations of LPS

FIG. 2: Survival curves of mice with sepsis in the prevention and treatment with different concentrations of ergothioneine

(panel A) treatment with different doses of ergothioneine; (panel B) prevention with different doses of ergothioneine

FIG. 3: Inflammatory factors in the blood of mice with sepsis at different time points after ergothioneine prevention

(panel A) IL-6; (panel B) TNF-α; (panel C) IL-1β

FIG. 4: Inflammatory factors in the blood of mice with sepsis at different time points after ergothioneine treatment

(panel A) IL-6; (panel B) TNF-α; (panel C) IL-1β

FIG. 5: Edema in the organs after ergothioneine prevention

(panel A) heart wet/dry weight ratio; (panel B) liver wet/dry weight ratio; (panel C) kidney wet/dry weight ratio; (panel D) intestine wet/dry weight ratio;

FIG. 6: Edema in the organs after ergothioneine treatment

(panel A) heart wet/dry weight ratio; (panel B) liver wet/dry weight ratio; (panel C) kidney wet/dry weight ratio; (panel D) intestine wet/dry weight ratio;

FIG. 7: Evaluation of the efficacy of ergothioneine in the treatment and prevention of lung injury in mice with sepsis

(panels A-C): inflammatory factor concentrations in BALF (IL-1β, IL-6 and TNF-α); (panel D) lung wet/dry weight ratio; (panel E) Evans blue analysis of vascular leakage; (panel F) changes in the protein content of BALF; (panel G) changes in the arterial partial pressure of oxygen in mice; (panel H) changes in the proportion of neutrophils in BALF;

FIG. 8: Related gene expression in intestinal tissues after ergothioneine treatment and prevention panel A: TLR-4; panel B: MyD88; panel C: NF-κB;

FIG. 9: Related gene expression in lung tissues after ergothioneine treatment and prevention panel A: TLR-4; panel B: MyD88; panel C: NF-κB;

FIG. 10: Characterization graphs of nanovesicles loaded with ergothioneine (panel A shows the morphology of the nanovesicles loaded with ergothioneine under a TEM microscope; panel B shows the particle size as measured by NTA and the potential; panel C shows the ergothioneine content of the prepared nanovesicles loaded with ergothioneine as measured by high performance liquid chromatography (HPLC));

FIG. 11. Evaluation of the efficacy of human umbilical cord mesenchymal stem cell-derived nanovesicles (HucMSC-NVs) loaded with ergothioneine in the treatment of sepsis-related acute lung injury (panels A-C show three classical inflammatory factors (IL-1β, IL-6 and TNF-α) in BALF); panel D shows the changes of protein content in BALF of mice.

### DETAILED DESCRIPTION

Ergothioneine, (S)-α-Carboxy-N,N,N-trimethyl-2-mercapto-1H-imidazole-4-ethanaminium inner salt Thioneine

Molecular formula: C9H15N302S

Molecular weight: 229.3

Melting point/melting point range: 255-259° C.

Appearance: white solid

Solubility: soluble in water, methanol and ethanol

The following test examples illustrated the use of ergothioneine in the treatment and prevention of sepsis diseases.

### Test Example

Objective: To explore whether ergothioneine has prophylactic and therapeutic effects on LPS-induced sepsis, and explore the relevant mechanism of action from changes in the concentration of inflammatory factors IL-6, IL-1B and TNF-α in serum and changes in the inflammation of the lungs, as well as related gene expression, so as to screen out drugs that can be widely used for the clinical treatment of sepsis.

Method: A mouse model of sepsis was induced by intraperitoneal injection of different toxic doses of 5 mg/kg, 10 mg/kg and 20 mg/kg LPS, and the effects of different doses of LPS on the survival rate of mice with sepsis were observed. A mouse model of sepsis was induced by intraperitoneal injection of a total lethal dose of 20 mg/kg LPS, and the effects of different concentrations of ergothioneine (5 mg/kg, 10 mg/kg, 20 mg/kg and 50 mg/kg) on the survival time of mice with sepsis were observed. The prevention group was given tail vein injections of ergothioneine 1 h before the establishment of the LPS-induced sepsis model; peripheral blood of the mice of the LPS-induced sepsis model was taken 2 h, 4 h, 6 h, 12 h and 24 h after the sepsis model was established by intraperitoneal injection of 20 mg/kg LPS, to observe the effect of ergothioneine on the concentration of IL-6, IL-1B and TNF-α in mouse serum. The treatment group was given tail vein injections of ergothioneine 2 h after the establishment of the model; peripheral blood of the mice of the LPS-induced sepsis model was taken 0.5 h, 4 h, 6 h, 12 h and 24 h after the sepsis model was established by intraperitoneal injection of 20 mg/kg LPS, to observe the effect of ergothioneine on the concentration of IL-6, IL-1B and TNF-α in mouse serum, and heart, liver, kidney and intestinal tissues were taken to observe the effect of ergothioneine on the extent of edema in the organs and on related gene expression; and ergothioneine treatment and prevention of sepsis-related acute lung injury in mice. Mice injected intraperitoneally with a placebo in place of LPS were used as a normal control group, and mice injected intraperitoneally with a placebo and given tail vein injections of ergothioneine were used as a drug control group.

Results: Survival rate was observed in mice with sepsis induced by intraperitoneal injection of different toxic doses of 5 mg/kg, 10 mg/kg and 20 mg/kg LPS: the survival rate of mice in the normal control group was 100%; the survival rate of the 5 mg/kg LPS group was 60%; the survival rate of the 10 mg/kg LPS group was 40%; the survival rate of the 20 mg/kg LPS group was 0. After treatment with different concentrations of ergothioneine, the survival time of mice with sepsis was increased: the average survival time of the 20 mg/kg LPS group was (28.8±6.22) h; the average survival time of the 5 mg/kg ergothioneine group was (41.4±10.48) h, (p<0.05); the average survival time of the 10 mg/kg ergothioneine group was (44.4±3.921) h, (p<0.05); the average survival time of the 50 mg/kg ergothioneine group was (46.8±2.68) h, (p<0.05).

After ergothioneine treatment or prevention, IL-6, TNF-α and IL-1β were all significantly reduced (p<0.05); after ergothioneine treatment, IL-6 and TNF-α were significantly reduced (p<0.05). The wet/dry weight ratios of the organs indicate that after intraperitoneal injection of 20 mg/kg LPS, intestinal wall edema (p<0.05) can be caused, aggregation of lung inflammation can be increased, pulmonary edema and protein leakage can be aggravated, the expression levels of the TLR-4, MyD88 and NF-κB genes can be significantly increased, and the TLR-4/MyD88/NF-κB signaling pathway can be activated; after ergothioneine treatment and prevention, intestinal wall edema (p<0.05) can be alleviated, lung inflammation can be alleviated, pulmonary edema, protein leakage and vascular injury can be alleviated, lung oxygenation can be improved, the expression of TLR-4, MyD88 and NF-κB genes can be reduced, and the TLR-4/MyD88/NF-κB signaling pathway can be inhibited.

Conclusion: Ergothioneine significantly increased the survival rate of mice with LPS-induced sepsis, extended their survival time, attenuated inflammatory factor release, and inhibited the TLR-4/MyD88/NF-κB signaling pathway.

### EXAMPLES

### Example 1. Determination of Effect on Survival Rate of Mice with Sepsis

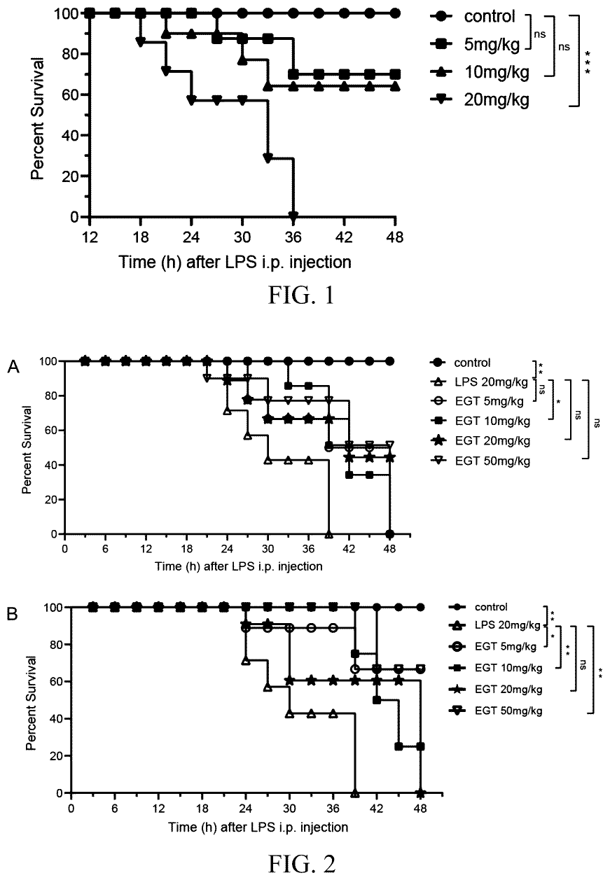

Sepsis was induced by intraperitoneal injection of 5 mg/kg, 10 mg/kg and 20 mg/kg LPS. The survival of mice is shown in FIG. 1: all in the control group survived; 2 mice in the 5 mg/kg group died; 3 mice in the 10 mg/kg group died; all in the 20 mg/kg group died. Sepsis was induced by intraperitoneal injection of 20 mg/kg LPS. Different doses of ergothioneine were used for prevention: 2 mice in the 5 mg/kg group died; 3 mice in the 10 mg/kg group died; all in the 20 mg/kg group died; 1 mouse in the 50 mg/kg died. Different doses of ergothioneine were used for treatment: all in the 5 mg/kg group died; all in the 10 mg/kg group died; 4 mice in the 20 mg/kg group died; 3 mice in the 50 mg/kg group died. Ergothioneine reduced mortality in mice with sepsis (FIG. 2).

### Example 2. Inflammatory Factors in Blood of Mice with Sepsis at Different Time Points After Ergothioneine Prevention

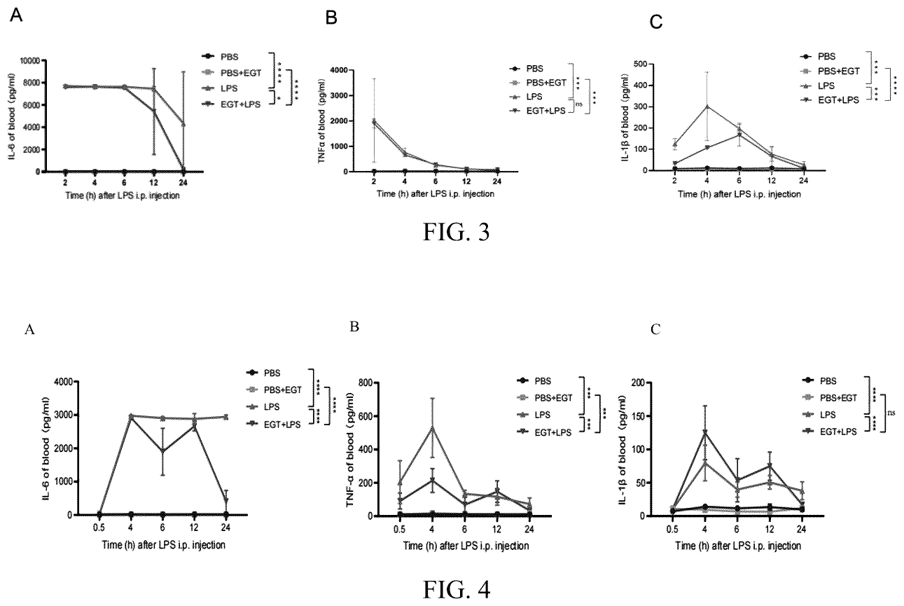

After ergothioneine prevention of LPS-induced sepsis, inflammatory factors at various time points are shown in FIG. 3. After LPS induction, inflammatory factors (IL-6, TNF-α and IL-1β) were significantly increased; after ergothioneine prevention, (IL-6 and IL-1β) release was significantly reduced, and the inflammatory factor storm was alleviated.

### Example 3. Inflammatory Factors in Blood of Mice with Sepsis at Different Time Points After Ergothioneine Treatment

After ergothioneine prevention of LPS-induced sepsis, inflammatory factors at various time points are shown in FIG. 4. After LPS induction, inflammatory factors (IL-6, TNF-α and IL-1β) were significantly increased; after ergothioneine prevention, (IL-6 and TNF-α) release was significantly reduced, and the inflammatory factor storm was alleviated.

### Example 4. Edema in Organs After Ergothioneine Prevention

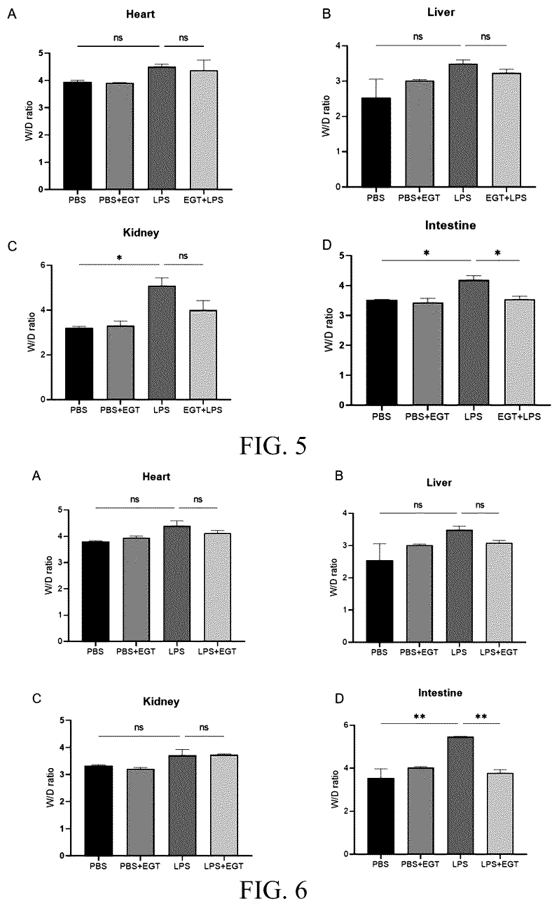

After ergothioneine prevention, edema in the organs was measured and is shown in FIG. 5. After LPS induction, intestinal wall edema was caused (W/D 4.182±0.204 vs 3.521±0.016, P<0.05), and kidney edema was caused (W/D 5.092±0.489 vs 3.197±0.103, P<0.05). After ergothioneine prevention, the extent of the intestinal wall edema was reduced (W/D 3.543±0.137 vs 4.182±0.204, P<0.05), but the kidney edema was not significantly ameliorated. After LPS induction of sepsis, no significant edema was seen in the heart and the liver.

### Example 5. Edema in Organs After Ergothioneine Treatment

After ergothioneine treatment, edema in the organs was measured and is shown in FIG. 6. After LPS induction, intestinal wall edema was caused (W/D 5.466±0.021 vs 3.550±0.418, P<0.05). After ergothioneine treatment, the extent of the intestinal wall edema was reduced (W/D 3.787±0.148 vs 5.466±0.021, P<0.05), and what made it different from the prevention group is that after LPS induction, no significant edema was seen in the kidneys, the heart and the liver.

### Example 6. Evaluation of Efficacy of Ergothioneine in Treatment and Prevention of Sepsis-Related Acute Lung Injury in Mice

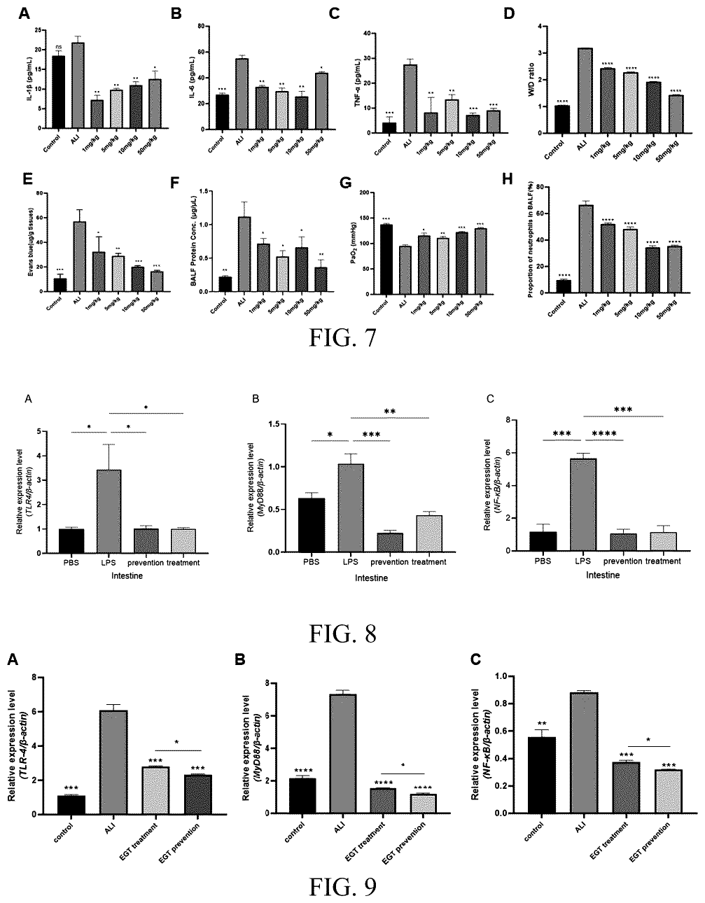

The evaluation of the efficacy of ergothioneine in the treatment and prevention of lung injury in mice with sepsis is shown in FIG. 7, where FIG. 7, panels A-C show three classical inflammatory factors (IL-1β, IL-6 and TNF-α) in bronchoalveolar lavage fluid (BALF) of mice. The data for the three inflammatory indicators (IL-1β, IL-6 and TNF-α) show that the four treatment groups using different concentrations of ergothioneine all reduced inflammatory factor levels in BALF and that even small doses of ergothioneine can have an effect. Comparisons of data between the four groups are of statistical significance.

Changes in the protein content of mouse BALF, the Evans blue content and the wet/dry weight ratio are shown. The results in FIG. 7, panels D-F show that even small doses of ergothioneine can effectively relieve leakage in the pulmonary capillaries of mice, and that leakage relief was more significant in the high-EGT-concentration group than in the other three groups; there is statistical significance between the four groups' data. FIG. 7, panel G shows the arterial partial pressure of oxygen in mice. There is a downward trend in the arterial partial pressure of oxygen in all the injury group mice relative to the normal mice. In the treatment of sepsis-related acute lung injury in mice with different concentrations of EGT, the arterial partial pressure of oxygen was significantly improved.

### Example 7. Related Gene Expression after Ergothioneine Treatment and Prevention

After ergothioneine prevention and treatment of sepsis in mice, the related gene expression in intestinal tissues was measured and is shown in FIG. 8 and Table 1: 20 mg/kg LPS significantly increased the expression of the TLR-4, MyD88 and NF-κB genes in intestinal tissues of mice (3.44 vs 1.004, P<0.05; 1.035 vs 0.633, P<0.05; 5.653 vs 1.182, P<0.05). After ergothioneine prevention, the expression of the TLR-4, MyD88 and NF-κB genes in intestinal tissues was significantly inhibited (1.012 vs 3.44, P<0.05; 0.432 vs 1.035, P<0.05; 1.065 vs 5.653, P<0.05). After ergothioneine treatment, the expression of the TLR-4, MyD88 and NF-κB genes in intestinal tissues was also significantly inhibited (1.003 vs 3.44, P<0.05; 0.227 vs 1.035, P<0.05; 1.141 vs 5.653, P<0.05).

TABLE 1
Related gene expression in intestinal tissues after ergothioneine
prevention and treatment of sepsis in mice

| | Control group | LPS group | Prevention group | Treatment group |
|---|---|---|---|---|
| | Difference fold | Difference fold | Difference fold | Difference fold |
| β-actin | 1.00 | 1.00 | 1.00 | 1.00 |
| TLR-4 | 1.004 | 3.44 | 1.012 | 1.003 |
| MyD88 | 0.633 | 1.035 | 0.432 | 0.227 |
| NF-κB | 1.182 | 5.653 | 1.065 | 1.141 |

After ergothioneine prevention and treatment of sepsis in mice, the related gene expression in lung tissues was measured and is shown in FIG. 9. As can be seen from FIG. 9, panels A-C, TLR-4, MyD88 and NF-κB in the lungs were significantly up-regulated after LPS induction. After ergothioneine treatment and prevention, the TLR-4, MyD88 and NF-κB genes can be significantly down-regulated. The differences are of statistical significance.

### Example 8. Preparation and Characterization of Human Umbilical Cord Mesenchymal Stem Cell-Derived Nanovesicles (HucMSC-NVs) Loaded with Ergothioneine

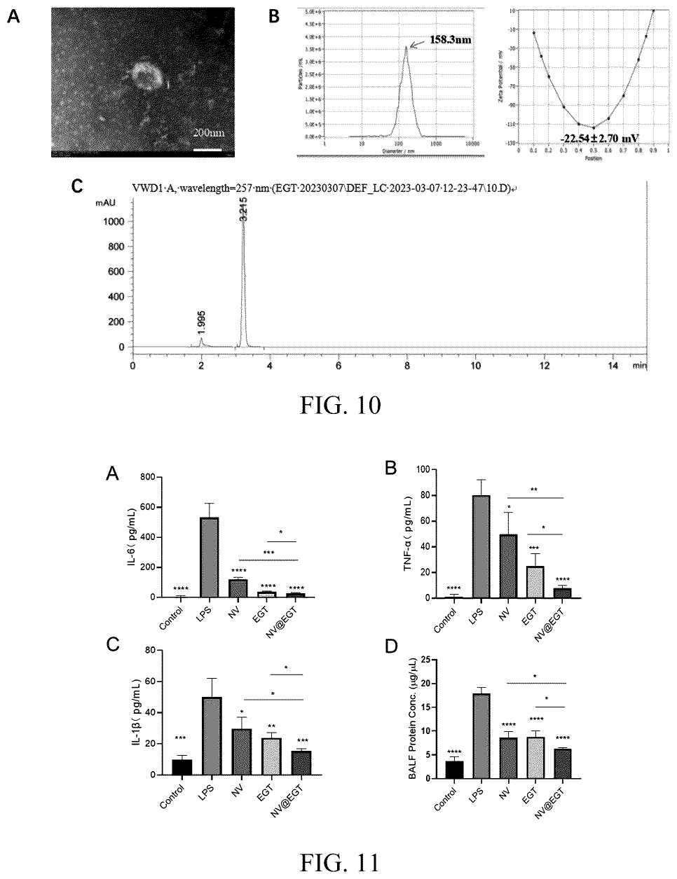

Preparation of nanovesicles loaded with ergothioneine: Human umbilical cord mesenchymal stem cells, together with ergothioneine, were extruded and continuously passed through polycarbonate membranes with different pore sizes to obtain nanovesicles loaded with ergothioneine. FIG. 10, panel A shows the morphology of the prepared nanovesicles loaded with ergothioneine under a TEM microscope; it can be seen from the TEM morphology that the prepared nanovesicles are within 200 nm in particle size. FIG. 10, panel B shows the particle size as measured by NTA and the potential; it can be seen that the particle size of the prepared nanovesicles loaded with ergothioneine focuses at 158.3 nm, and that the potential is −22.54±2.70 mV. The results in FIG. 10, panels A and B indicate the successful preparation of nanovesicles loaded with ergothioneine. FIG. 10, panel C shows the ergothioneine content of the prepared nanovesicles loaded with ergothioneine as measured by high performance liquid chromatography (HPLC). The drug-loading capacity can be calculated at 32.6 ng/μg (EGT/HucMSC-NVs).

### Example 9. Evaluation of Efficacy of Human Umbilical Cord Mesenchymal Stem Cell-Derived Nanovesicles (HucMSC-NVs) Loaded with Ergothioneine in Treatment of Sepsis-Related Acute Lung Injury

Referring to FIG. 11, three classical inflammatory factors (IL-1β, IL-6, and TNF-α) in BALF are shown in panels A-C. The data of the three groups of inflammatory indicators (IL-6, TNF-α, IL-1β) showed that all reduced the level of inflammatory factors in BALF, and the HucMSC-NVs-loaded ergothioneine treatment group was more effective compared to the HucMSC-NVs-treated group with ergothioneine treatment group, and the comparison between the data of all five experimental groups was statistically significant. Panel D shows the changes of protein content in BALF of mice. Similarly, the HucMSC-NVs-loaded ergothioneine treatment group improved capillary leakage in mice compared with the HucMSC-NVs treatment group and ergothioneine treatment group.

1. A method of prevention or treatment of sepsis or related disease in a subject, wherein the method comprises administering ergothioneine and/or a pharmaceutically acceptable derivative thereof to the subject.

2. The method as claimed in claim 1, wherein the sepsis-related disease selects from septic shock or sepsis-induced organ injury.

3. The method as claimed in claim 1, wherein the prevention or treatment includes amelioration of a disease symptom or improvements in health in the subject.

4. The method as claimed in claim 3, wherein the prevention or treatment alleviates lung inflammation, alleviates pulmonary edema, protein leakage and vascular injury, and improves lung oxygenation.

5. The method as claimed in claim 4, wherein the prevention or treatment inhibits phosphorylation activation of the TLR-4/MyD88/NF-κB signaling pathway.

6. The method as claimed in claim 1, wherein the ergothioneine and/or the pharmaceutically acceptable derivative thereof are/is used concurrently with one or more other drugs for the prevention or treatment of sepsis or the related disease.

7. The method as claimed in claim 1, wherein the ergothioneine and/or the pharmaceutically acceptable derivative thereof are/is administered by intraperitoneal injection, intramuscular injection or intravenous injection, or are/is administered orally.

8. The method as claimed in claim 7, wherein the ergothioneine and/or the pharmaceutically acceptable derivative thereof are/is administered in an amount of 0.1-150 mg/kg.

9. The method as claimed in claim 8, wherein the ergothioneine and/or the pharmaceutically acceptable derivative thereof are/is administered in an amount of 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145 or 150 mg/kg, based on ergothioneine.

10. The method as claimed in claim 7, wherein the ergothioneine or the pharmaceutically acceptable salt thereof is a solid or liquid preparation in a unit dosage form.

11. The method as claimed in claim 10, wherein the content of ergothioneine or the pharmaceutically acceptable salt thereof in the unit dosage form is 0.1-1000 mg, based on ergothioneine.

12. The method as claimed in claim 11, wherein the content of ergothioneine or the pharmaceutically acceptable salt thereof in the unit dosage form is 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 550, 600, 650, 700, 750, 800, 850, 900, 950 or 1000 mg, based on ergothioneine.

13. The method as claimed in claim 7, wherein a nano-sized pharmaceutical carrier loaded with ergothioneine is used to treat or prevent sepsis, wherein the nano-sized pharmaceutical carrier selects from organic nanoparticles, inorganic nanoparticles, and nanoparticles of natural origin; the organic nanoparticles select from liposomes, micelles, emulsions and solid lipid nanoparticles (SLNs) and polymeric nanoparticles (PNPs); the inorganic nanoparticles (INPs) are synthesized from inorganic particles and biodegradable polycations, and select from metals, metal oxides, carbon materials, and magnetic nanoparticles consisting of superparamagnetic iron oxide nanoparticles (SPIONs); the nanoparticles of natural origin select from extracellular vesicles (EVs) actively released by cells and nanovesicles (NVs) prepared by artificial extrusion.

14. The method as claimed in claim 13, wherein the extracellular vesicles (EVs) select from exosomes or microvesicles.

15. The method as claimed in claim 1, wherein the pharmaceutically acceptable derivative of ergothioneine selects from one or more of a pharmaceutically acceptable salt of ergothioneine, a tautomer of ergothioneine and a stereoisomer of ergothioneine.

16. The method as claimed in claim 1, wherein the subject is a human.

17. A nanodrug, wherein the drug comprises a nano-sized pharmaceutical carrier, and ergothioneine and/or a pharmaceutically acceptable derivative thereof loaded on the carrier, wherein the nano-sized pharmaceutical carrier selects from organic nanoparticles, inorganic nanoparticles and nanoparticles of natural origin.

18. The nanodrug as claimed in claim 17, wherein the organic nanoparticles select from liposomes, micelles, emulsions and solid lipid nanoparticles (SLNs) and polymeric nanoparticles (PNPs); the inorganic nanoparticles (INPs) are synthesized from inorganic particles and biodegradable polycations, and select from metals, metal oxides, carbon materials, and magnetic nanoparticles consisting of superparamagnetic iron oxide nanoparticles (SPIONs); the nanoparticles of natural origin select from extracellular vesicles (EVs) actively released by cells and nanovesicles (NVs) prepared by artificial extrusion.

19. The nanodrug as claimed in claim 18, wherein the extracellular vesicles (EVs) select from exosomes or microvesicles.
